(19) United States
(12) Patent Application Publication
(10) Pub. No.: US 2007/0173926 A1
(43) Pub. Date: Jul. 26, 2007

(54) Anchoring system for medical implant

(76) Inventors: Donald E. Bobo JR., Santa Ana, CA (US); Henry Bourang, Irvine, CA (US); Philip P. Corso JR., Newport Coast, CA (US); William R. Wertenberg, Newport Beach, CA (US); Mark Chau, Camarillo, CA (US); Michael Popp, Orange, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/637,492

(22) Filed: Dec. 11, 2006

Related U.S. Application Data
(60) Provisional application No. 60/749,215, filed on Dec. 9, 2005.

(51) Int. Cl. A61F 2/06

(52) U.S. Cl. 623/1.16

(57) ABSTRACT

Embodiments of the invention provide a medical implant, and method for deploying the same, configured for placement in a body lumen such as a coronary sinus, as may be desired to treat a regurgitant mitral valve. The implant includes a proximal anchor, a distal anchor, and an elongate bridge extending between the proximal and distal anchors. The anchors are configured to be delivered to a desired deployment site within the body lumen in a collapsed or contracted condition and then expanded to engage the walls of the body lumen. The bridge is preferably a shape-changing member configured to contract after delivery into the coronary sinus for applying a compressive force to the annulus of the mitral valve. The proximal and distal anchoring mechanisms preferably overlap the proximal and distal end portions of the bridge, thereby providing the implant with a longer bridge having greater contraction.

### RELATED APPLICATIONS

The present invention claims priority to Provisional Application No. 60/749,215, filed on Dec. 9, 2005, entitled “Device and Method For Treating a Mitral Valve.”

### FIELD OF THE INVENTION

The present invention relates to a medical implant, and more particularly to a medical implant configured to reshape the annulus of a mitral valve.

### BACKGROUND OF THE INVENTION

Heart valve regurgitation, or leakage from the outflow to the inflow side of a heart valve, is a condition that occurs when a heart valve fails to close properly. Regurgitation through the mitral valve is often caused by changes in the geometric configurations of the left ventricle, papillary muscles, and mitral valve annulus. Similarly, regurgitation through the tricuspid valve is often caused by changes in the geometric configurations of the right ventricle, papillary muscles, and tricuspid annulus. These geometric alterations can result in incomplete coaptation of the valve leaflets during systole.

A variety of heart valve repair procedures have been proposed over the years for treating defective heart valves. With the use of current surgical techniques, it has been found that many regurgitant heart valves can be repaired.

In recent years, several new minimally invasive techniques have been introduced for repairing defective heart valves wherein open-heart surgery and cardiopulmonary by-pass are not required. Some of these techniques involve introducing an implant at least partially into a coronary sinus for reshaping the mitral valve annulus. The mitral valve annulus consists of a ring of collagenous tissue that surrounds and supports the mitral valve leaflets. The coronary sinus is a blood vessel that extends around a portion of the heart through the atrioventricular groove in close proximity to the posterior, lateral, and medial aspects of the mitral valve annulus. Because of its position, the coronary sinus provides an ideal conduit for receiving an implant (i.e., endovascular device) configured to apply a reshaping force from within the coronary sinus to effect the shape of the mitral valve annulus. Various examples of mitral valve repair devices which are configured for insertion into the coronary sinus are described in Applicant's U.S. Publication No. 2005/0177228, filed Dec. 15, 2004, the entire contents of which are incorporated herein by reference.

In one configuration, an implant for treating mitral regurgitation includes a proximal anchor, a distal anchor, and an elongate bridge portion extending between the proximal and distal anchors. The proximal and distal anchors are secured to the inner walls of the coronary sinus and the bridge portion foreshortens over time, thereby applying a reshaping force to the annulus of the mitral valve. This force reshapes the geometry of the mitral valve for the purpose of improving coaption of the mitral valve leaflets and reducing or eliminating mitral vale leakage. Although medical implants of this type are effective in treating mitral regurgitation, it has been found that the coronary sinus and mitral valve annulus can vary substantially in anatomical structure. As a result, a need exists for an improved device having a more flexible and adaptable connection between the bridge and anchors. The present invention addresses this need.

### SUMMARY OF THE INVENTION

Preferred embodiments of the present invention provide an implant, and method of use therefore, configured for placement in a body lumen such as the coronary sinus. The implant has a first anchor, a second anchor, and an elongate bridge portion that is secured to the first and second anchors. The first and second anchors are configured to radially expand into contact with the walls of the body lumen so that the anchors are secured within the body lumen. After deployment in a coronary sinus of a heart, the implant changes shape to apply a reshaping force along the coronary sinus axis and the posterior portion of a mitral annulus. The applied force restores proper mitral valve leaflet coaptation and thereby reduces or eliminates mitral valve regurgitation.

In one preferred aspect of the present invention, an implant for treating mitral valve annulus dilatation comprises a bridge in the form of a shape-changing member having a proximal end portion and a distal end portion. The shape-changing member has first shape and a second shape. A displaceable or removable material is disposed along the shape-changing member for temporarily maintaining the shape-changing member in the first shape. The displaceable material is configured to be displaced for allowing the shape-changing member to transition from the first shape to the second shape after implantation in the coronary sinus. A proximal anchor is coupled to the proximal end portion of the shape-changing member and a distal anchor is coupled to the distal end portion of the shape-changing member. In an advantageous feature, the proximal and distal anchors are configured with improved structures such that the proximal end portion of the shape-changing member overlaps with at least a portion of the proximal anchor and the distal end portion of the shape-changing member overlaps with at least a portion of the distal anchor. Because the shape-changing member overlaps the anchors, the shape-changing member comprises a larger portion of the overall length of the implant, thereby increasing the effectiveness and adaptability of the implant.

In one variation, the shape-changing member is coupled to the proximal and distal anchors by suture. More particularly, the proximal and distal ends of the shape-changing member are tied to the proximal and distal anchors, respectively. Preferably, only the ends of the shape-changing member are attached to the anchors such that the remaining portion of the shape-changing member can slide relative to the anchors at it contracts. In other variation, mechanisms such as wire or polymers may be used as coupling members.

In another variation, the shape-changing member is flexibly coupled to the proximal and distal anchors, such as by one or more flexible mechanical linkages. In preferred embodiments, the mechanical linkages exhibit sufficient flexibility for reducing stress concentrations at the attachment points.

In another variation, the shape-changing member and the proximal and distal anchors are integrally formed from a single piece of material during construction. For example, the components of the implant may be laser cut from a sheet of material and then shaped, rolled or folded into the desired configuration. Alternatively, the anchors and shape-changing member may be constructed separately and then joined together to form the implant. In either case, the proximal and distal anchors are preferably constructed to self-expand after being released from a delivery sheath.

In another variation, the proximal and distal anchors comprise proximal and distal stents. In one embodiment, the distal end of the proximal stent and the proximal end of the distal stent have curvilinear shapes such that a first wall of each stent has a first longitudinal length and a second wall of each stent has a second longitudinal length which is longer than the first length and wherein the shape-changing member is attached to the first wall. By attaching the shape-changing member to the shorter wall of the stent, the shape-changing member may have a longer length. In another embodiment, the proximal end portion of the shape-changing member extends through an interior region of the proximal stent and the distal end portion of the shape-changing member extends through an interior region of the distal stent. In other words, the shape-changing member passes through the stents and the shape-changing member is preferably fixedly attached to a proximal end of the proximal stent and to a distal end of the distal stent.

In another variation, the proximal and distal anchors comprise stents formed with longitudinal slots. The longitudinal slots are configured for receiving the proximal and distal end portions of the shape-changing member. The ends of the shape-changing member are preferably fixed to the stents while the end portions of the shape-changing member extending through the slots are slidably engaged to the stent. Coupling members are provided for allowing the shape-changing member to move relative to the anchors during contraction, while maintaining the components in a desired alignment.

In another variation, barbs or other engagement members are disposed along the proximal and distal end portions of the shape-changing member. The barbs are configured for engaging tissue within the coronary sinus to more securely anchor the ends of the shape-changing member to the coronary sinus.

In another variation, at least one of the proximal and distal stents has a flared end region for improved anchoring.

In another variation, the shape-changing member is rotatably or hingedly coupled to at least one of the proximal and distal anchors. A rotatable or hinged attachment allows articulation of the shape-changing member relative to the anchors such that the shape-changing member and anchors can move semi-independently. This feature advantageously allows the implant to conform to tortuous regions of the coronary sinus without creating stress concentrations at the attachment points.

In another preferred aspect of the present invention, a medical implant comprises a proximal anchor configured for engagement to an ostium of a coronary sinus when in a deployed position, a distal anchor configured for engagement with an inner wall of a coronary sinus when in a deployed position, and an elongate bridge extending between the proximal and distal anchors, the elongate bridge configured for applying a reshaping force along an annulus of a mitral valve. The proximal and distal anchors are preferably capable of pivoting relative to the elongate bridge along at least one axis. This feature allows the bridge to extend away from the anchors at a different relative angle and thereby reduces or eliminates stress concentrations at the attachment points. This type of coupling also advantageously allows the anchors and bridge to move semi-independently of each other. Preferably, the elongate bridge is formed of a shape-memory material and the bridge is maintained in an elongated state by a resorbable material during implantation. The bridge is biased to transition to a contracted state as the resorbable material is gradually resorbed after implantation.

In another preferred aspect of the present invention, a medical implant for treating a mitral valve comprises a proximal stent configured for engagement to an ostium of a coronary sinus when in an expanded condition, a distal stent configured for engagement with an inner wall of a coronary sinus when in an expanded condition, and an elongate bridge coupled to the proximal and distal stents, the elongate bridge formed of a shape-memory material having a proximal end portion which overlaps with the proximal stent and a distal end portion which overlaps with the distal stent. The bridge is configured to contract after the proximal and distal stents are anchored within the coronary sinus such that the resulting tension in the bridge provides a reshaping (i.e., shape-changing) force along a posterior region of a dilated mitral valve annulus. Because the bridge overlaps with the proximal and distal stents, the bridge extends along a greater percentage of the overall implant length. In one preferred configuration, the bridge has a length which is greater than 90% of a total length of the implant. In another preferred configuration, the length of the bridge is substantially equal to a total length of the implant.

Other objects, features, and advantages of the present invention will become apparent from a consideration of the following detailed description.

### BRIEF DESCRIPTION OF THE DRAWINGS

FIG. 1 is a three-dimensional view illustrating a mitral valve and coronary sinus which form a portion of a human heart.

FIG. 2 is a side view of a medical implant configured for delivery into a coronary sinus comprising a bridge and proximal and distal anchors.

FIG. 3 is an enlarged schematic view of the bridge of FIG. 2.

FIG. 4 is a plan view illustrating an improved medical implant having an anchoring mechanism configured such that the ends of the bridge are embedded into the proximal and distal anchors;

FIG. 5 is an enlarged view illustrating the connection of the bridge to the distal anchor of the implant of FIG. 4;

FIG. 6 illustrates a preferred delivery system configured for use with the implant of FIG. 4;

FIG. 7 is an enlarged view illustrating an alternative configuration for coupling a bridge to an anchor;

FIG. 8 is a perspective view illustrating another configuration for embedding a bridge into an anchor;

FIG. 8A is a perspective view illustrating a variation of FIG. 8 wherein the bridge is attached to the anchor using a rotatable coupling member;

FIG. 8B is a perspective view illustrating another variation of FIG. 8 wherein the bridge is secured to a cut-away stent, wherein the stent shape allows for greater articulation of the bridge;

FIG. 9 is a perspective view illustrating yet another preferred configuration wherein a proximal end of a bridge is fixed to a proximal end of a proximal anchor and a distal end of the bridge is fixed to a distal end of a distal anchor;

FIG. 10 is a perspective view illustrating yet another preferred configuration of an implant having anchors formed with slots for receiving a bridge;

FIG. 10A is a perspective view illustrating the distal anchor used with the implant of FIG. 10;

FIG. 10B is a perspective view illustrating the connection between the distal anchor and the bridge for the implant of FIG. 10;

FIG. 11 illustrates another preferred configuration wherein an end portion of a bridge is fixed to an anchor and another portion of the bridge is slideably attached to the anchor;

FIG. 12 illustrates yet another preferred configuration wherein barbs are provided along proximal and distal ends of a bridge to further enhance the anchoring mechanism; and

FIG. 13 illustrates yet another preferred configuration wherein end portions of proximal and distal anchors are flared to a larger diameter.

### DETAILED DESCRIPTION OF THE PREFERRED EMBODIMENTS

Various embodiments of the present invention depict medical implants and methods of use that are well-suited for treating mitral valve regurgitation. However, it should be appreciated that the principles and aspects of the embodiments disclosed and discussed herein are also applicable to other devices having different structures and functionalities. For example, certain structures and methods disclosed herein may also be applicable to other medical devices configured for implantation in a blood vessel. Furthermore, certain aspects of the present invention may also be used in conjunction with other medical devices or other procedures not explicitly disclosed. The manner of adapting the embodiments described herein to various other devices and functionalities will become apparent to those of skill in the art in view of the description that follows.

As used herein, “distal” means the direction of a device as it is being inserted into a patient's body or a point of reference closer to the leading end of the device as it is inserted into a patient's body. Similarly, as used herein “proximal” means the direction of a device as it is being removed from a patient's body or a point of reference closer to a trailing end of the device as it is inserted into a patient's body.

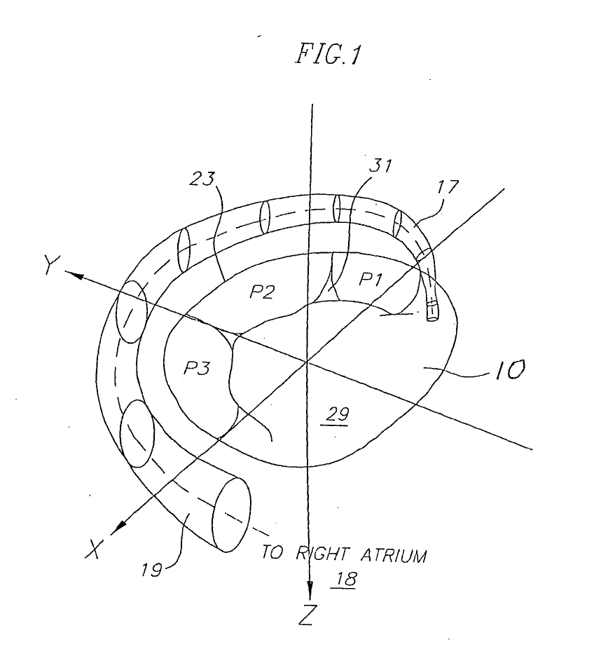

With reference now to FIG. 1, a three-dimensional view of a mitral valve 10 and a coronary sinus 17 is shown. From this view, it can be seen that the coronary sinus extends around a posterior region of the mitral valve 10. The coronary sinus is a relatively large vessel that receives venous drainage from the heart muscle. Blood flows through the coronary sinus and empties into the right atrium 18 through a coronary ostium 19. A mitral valve annulus 23 is a portion of tissue surrounding a mitral valve orifice to which the valve leaflets attach. The mitral valve 10 has two leaflets, an anterior leaflet 29 and a posterior leaflet 31. The posterior leaflet has three scallops P1, P2 and P3. As used herein, the term coronary sinus 17 is used as a generic term that describes the portion of the vena return system that is primarily situated adjacent to the mitral valve 10 and extends, at least in part, along the atrioventricular groove. Accordingly, the term coronary sinus 17 shall be understood to include the great cardiac vein and all other related portions of the vena return system.

Dilation of the mitral valve annulus 23 and/or dislocation of the valve leaflets are the primary causes of regurgitation through the mitral valve 10. More particularly, when a posterior aspect of the mitral valve annulus 23 dilates or when the leaflets are pulled out of alignment due to a dilating ventricle, one or more of the posterior leaflet scallops P1, P2, P3 moves away from the anterior leaflet 29. As a result, the anterior and posterior leaflets of the mitral valve fail to close completely during ventricular systole and blood flows backward (i.e., regurgitates) through the resulting gap. To reduce or eliminate mitral regurgitation, it is desirable to move the posterior aspect of the dilated mitral valve annulus 23 in an anterior direction or re-establish proper leaflet geometry, thereby narrowing or closing the gap between the leaflets.

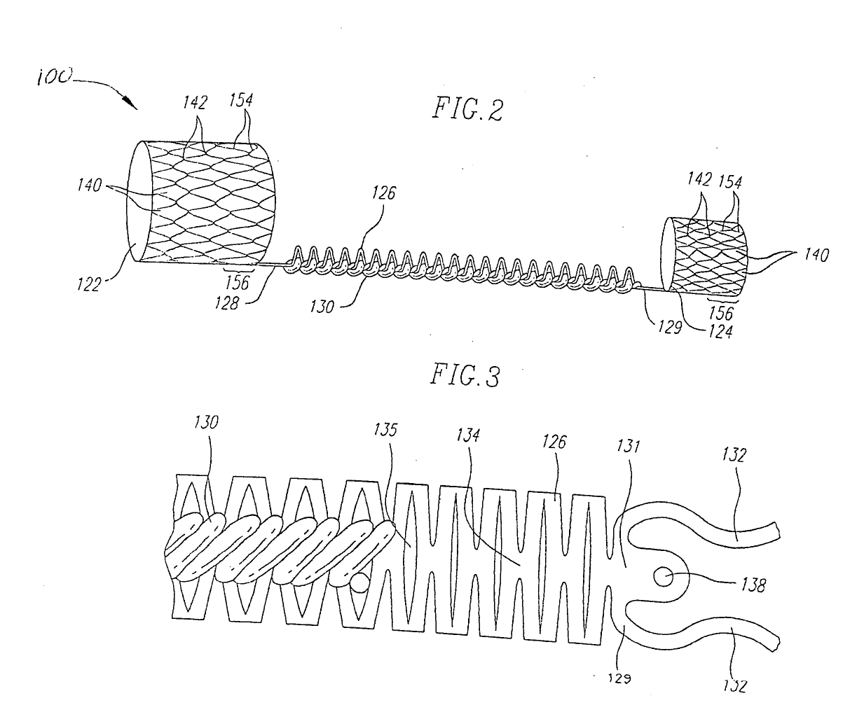

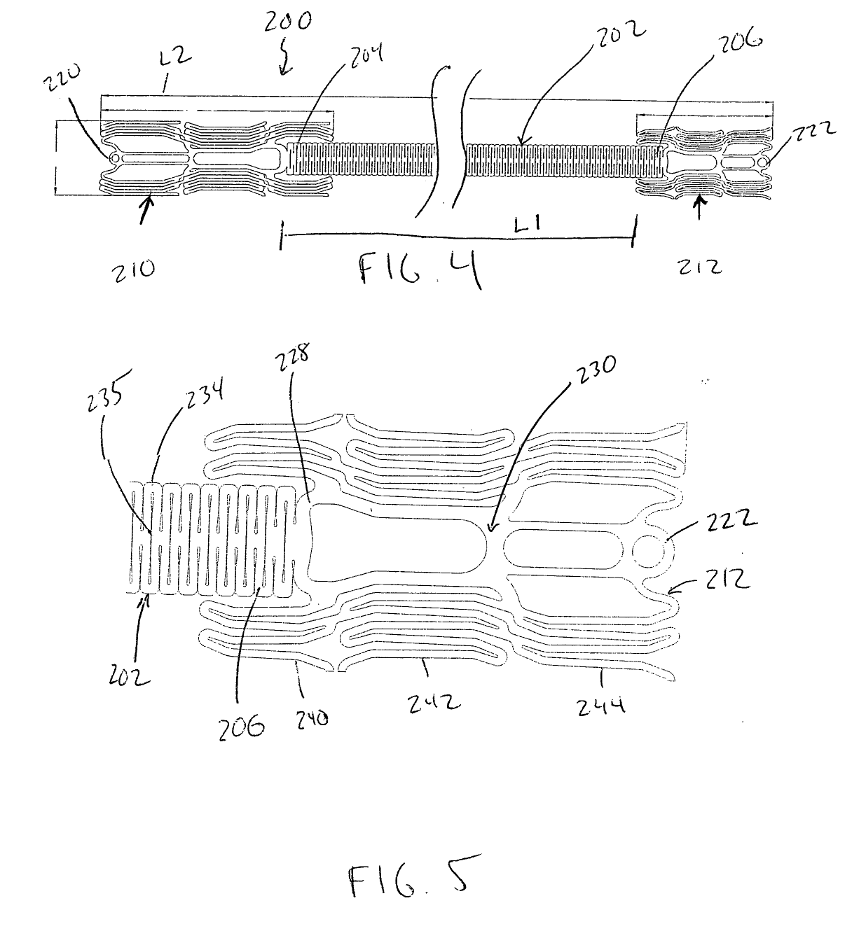

With reference now to FIGS. 2 and 3, one known configuration of a mitral valve repair implant 100 is illustrated. The implant is sized for deployment in the coronary sinus and is configured to apply a reshaping force along the axis of the coronary sinus and along the posterior portion of the mitral annulus. As illustrated, the implant 100 includes a proximal anchor 122 and a distal anchor 124 connected by a bridge 126. The bridge 126 is configured to foreshorten after the proximal and distal anchors are secured within the coronary sinus. A resorbable material is disposed within openings 135 in the bridge.

The resorbable material maintains the bridge in a stretched length during delivery and deployment. Over time, the resorbable material is resorbed and the bridge returns to its relaxed (i.e., shortened) length. As the bridge shortens, it tightens against the posterior aspect of the mitral valve annulus for reducing dilation of the mitral valve annulus. Additional details regarding medical implants and preferred methods of use for treating mitral valve regurgitation may be found in Assignee's U.S. Pat. No. 6,210,432, U.S. Pat. No. 6,997,951, U.S. Pat. No. 7,090,695, U.S. application Ser. No. 10/141,348, filed May 9, 2002, and U.S. application Ser. No. 11/238,853, filed Sep. 28, 2005, each of which is hereby incorporated by reference in its entirety.

With continued reference to the embodiment illustrated in FIGS. 2 and 3, the proximal and distal anchors 122, 124 are both cylindrical in shape and are formed from tubes of shape memory material, such as, for example, Nitinol. In the illustrated embodiment, both anchors 122, 124 have a mesh configuration comprising loops 154 of zig-zag shaped shape memory material having alternating peaks 142. The loops 154 are connected at each peak 142 to form rings 156 of four-sided openings 140.

The bridge 126 is connected to the proximal anchor 122 and distal anchor 124 by proximal and distal links 128, 129. More specifically, as shown in FIG. 2, the proximal link 128 connects the proximal anchor 122 to a proximal end of the bridge 126 and the distal link 129 connects the distal anchor 124 to a distal end of the bridge 126. In the illustrated embodiment, each of the links 128, 129 has a base 131 and arms 132 that extend from the base. The arms are connected to peaks 142 on each anchor 122, 124. Further, the links 128, 129 may be provided with a hole 138, as shown in FIG. 3, which serves as a means through which to pass an end of the resorbable thread and secure it to the bridge 126.

With continued reference to the embodiment illustrated in FIGS. 2 and 3, the bridge 126 is formed with a plurality of expandable elements (or cells) 134. In the illustrated embodiment, each expandable element 134 generally comprises an X-shaped member, wherein each X-shaped member is connected to an adjacent X-shaped member at the extremities of the “X.” The connection of the X-shaped members creates a plurality of openings 135 between the expandable elements 134. As best shown in FIG. 3, the openings are larger when the bridge is in a stretched condition. If desired, the X-shaped members may be formed with rounded edges that minimize the chance that a sharp edge of the bridge 126 will damage the coronary sinus 17 during delivery of the implant 100.

In the illustrated embodiment, the resorbable thread 130 is woven into the openings 135 (as shown in FIGS. 2 and 3) between adjacent expandable elements 134. The thread acts as a temporary spacer which prevents the openings from contracting. Accordingly, the thread temporarily maintains the bridge 126 in its stretched condition. As the resorbable thread 130 dissolves over time, the openings 135 contract (i.e., become more narrow in width). As a result, the bridge gradually reduces in length and pulls on the proximal and distal anchors. Because the proximal and distal anchors are secured within the coronary sinus, the contraction of the openings increases the tension in the bridge. When the implant is initially deployed, the bridge follows a curved path (i.e., along the curvature of the coronary sinus). However, the increased tension causes the bridge to adjust toward a straighter path. As the shape of the bridge straightens, the bridge applies a reshaping force along the posterior portion of the dilated mitral valve annulus, thereby reshaping the mitral valve annulus and reducing mitral regurgitation.

As discussed above, the implant is configured such that contraction of the bridge increases the reshaping force on the posterior portion of the mitral valve annulus. The compressive force reshapes the mitral valve annulus and improves the function of the mitral valve leaflets. However, using the implant described above with reference to FIGS. 2 and 3, it has been found the amount of bridge contraction may not be sufficient to adequately treat a mitral valve in all cases nor does it provide the necessary adaptability to allow the bridge to follow the inside curvature of the coronary sinus. A longer bridge would result in greater contraction and therefore greater effectiveness; however, the overall length of the implant is limited by the anatomy of the coronary sinus. It would be possible to increase the length of the bridge by shortening the lengths of the anchors; however, shorter anchors may not be desirable because they may lack the ability to adequately secure the implant to the vessel. Furthermore, it has been found that the human anatomy of the coronary sinus has curvatures that would benefit from a more flexible and adaptive connection between the anchor and the bridge. More particularly, a bridge that extends into the proximal or distal anchor would allow the bridge to follow a more aggressive curvature angle into the coronary sinus without being constrained by the anchor. Such a connection could also reduce the stress concentrations which sometimes result from the bent and/or twisted position of the implant after deployment in the coronary sinus.

Accordingly, there is a need for an improved mitral valve repair implant having an alternative anchoring mechanism which allows the use of a longer bridge without sacrificing the integrity and effectiveness of the anchors. There is also a need for an improved implant having more flexible connections between the bridge and anchors for conforming to the tortuous anatomy of the coronary sinus and reducing stress concentrations at the attachment points. As will be discussed in more detail below, this need is addressed by new and improved medical implants having anchoring mechanisms which allow the bridge length to be increased and/or which allow the bridge and anchors to move semi-independently of each other.

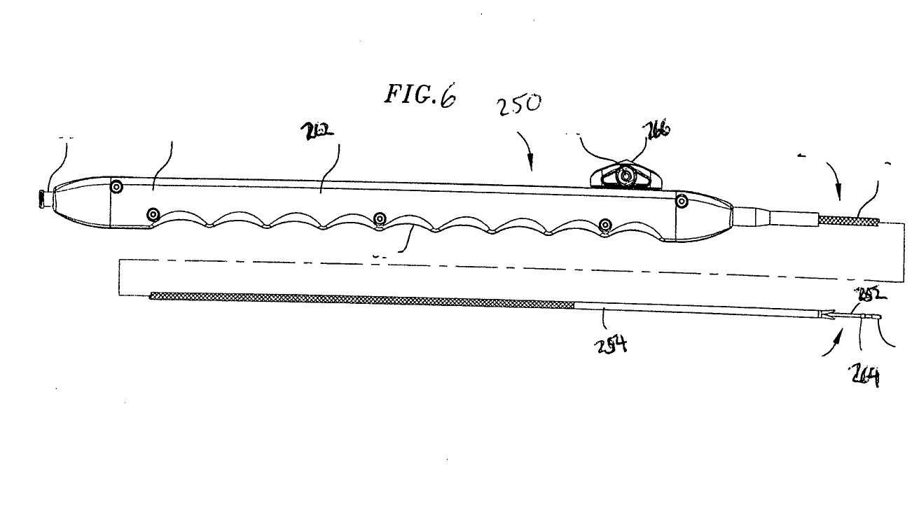

With reference now to FIG. 4, for purposes of illustration, a plan view of an improved mitral valve repair implant 200 is provided in accordance with one preferred embodiment of the present invention. The implant is preferably formed at least in part of a shape memory material and is configured for reshaping a dilated mitral valve annulus as generally described above. Although the implant is described with respect to treating mitral valves, the features of the implant may also be applied to other treatments, such as treatment of the tricuspid valve.

The mitral valve repair implant 200 generally comprises an elongate bridge 202 which provides a shape-changing member that is configured to contract and/or bend after placement in the coronary sinus. The implant further comprises proximal and distal anchors 210, 212 which are coupled to the proximal and distal end portions 204, 206 of the bridge. In an important feature, the proximal and distal end portions of the bridge 202 are “embedded” into the proximal and distal anchors 210, 212 such that at least a portion of each anchor overlaps with a portion of the bridge. More particularly, the proximal end of the bridge is located between the proximal and distal ends of the proximal anchor and the distal end of the bridge is located between the proximal and distal ends of the distal anchor. The bridge is embedded into the anchors such that the bridge is provided with a longer and more flexible construction without increasing the overall length L2 of the elongate body. The location and construction of the attachment points are also preferably configured to better distribute stresses and thereby enhance the structural integrity of the implant. In preferred embodiments, the length L1 of the bridge 202 comprises more than 70% of the total length L2 of the implant. More preferably, the length L1 of the bridge 202 comprises more than 90% of the total length L2 of the implant.

In preferred embodiments, the configuration illustrated in FIG. 4 is cut from a single piece of material such that the bridge 202, the proximal anchor 210 and distal anchor 212 are formed as an integral unit. However, in alternative constructions, the bridge and anchors may be formed separately and then attached. The proximal and distal anchors 210, 212 illustrated in FIG. 4 are shown laid out flat for ease of illustration. However, during use, the material comprising the proximal and distal anchors is wrapped to provide generally cylindrical elongate bodies (e.g., proximal and distal stents) configured for engaging the inner wall of a coronary sinus.

The bridge 202 is preferably formed of a shape memory material, such as, for example, Nitinol, and is preferably flexible in construction such that it is able to conform to a shape of the coronary sinus. The bridge 202 has two states: an elongated state in which the bridge has a first axial length, and a shortened state, in which the bridge has a second axial length, the second axial length being shorter than the first axial length. The bridge 202 is preferably biased toward the shortened state such that tension in the bridge increases after placement in the body. The bridge gradually returns to the shortened state as the resorbable material is resorbed over time (as generally described above). This “delayed memory” effect advantageously allows the proximal and distal anchors to securely attach to the coronary sinus before the bridge shortens. The delayed memory effect also provides the heart with time to gradually adjust to the reshaping of the mitral valve annulus over a variety of conditions (e.g., high and low blood pressures, etc.). As a result of the gradual adjustment, leaflet coaption is improved and the reduction in mitral regurgitation is enhanced.

Although the bridge configuration described herein is preferably used with a resorbable material, it will be recognized by those skilled in the art that the advantages and features of the improved anchoring mechanism may be applied to other implants configurations. For example, features of the anchoring mechanism described herein may be used with an “acute cinching” device wherein the distal anchor is deployed and the implant is then pulled proximally to tighten the bridge (and thereby reshape the mitral valve annulus) before the proximal anchor is deployed. Still further, a hybrid approach may be used wherein the distal anchor of an implant with a contractible bridge is deployed and the proximal anchor is pulled to partially reshape the annulus in an acute manner. After the proximal anchor is deployed, the delayed contraction of the bridge would then further reshape the annulus as the resorbable material is resorbed by the body. In another variation, the implant may be formed with a displaceable material for maintaining the bridge in the elongated state. A displaceable material could take the form any material which could be disposed along the bridge and then later displaced (e.g., removed) for allowing the bridge to shorten. In various examples, the displaceable could be mechanically removed or detached from the implant.

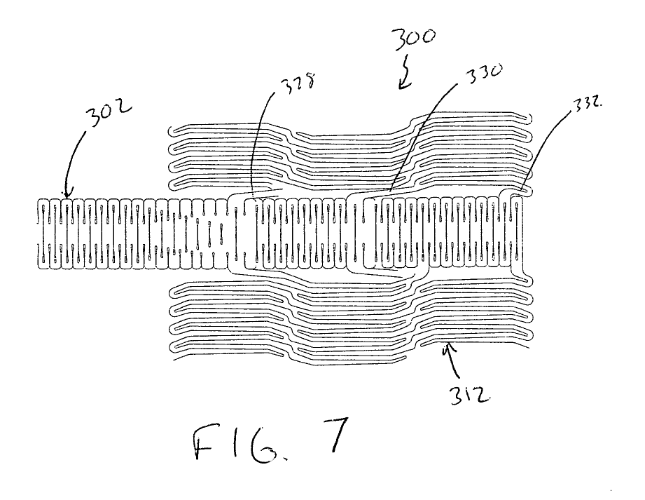

With reference to FIG. 5, the distal end portion of the implant 200 is shown in more detail. The bridge 202 is preferably formed with a plurality of expandable elements 234, each element being generally 0-shaped when in an expanded condition and each having an opening 235 through which a resorbable material, such as, for example, a thread, may be woven, sprayed, pressed or otherwise inserted. Each element 234 is attached to an adjacent element to form the bridge 202. As shown in FIGS. 4 and 5, the elements 234 are preferably integrally formed and may all be cut from a single piece of material.

In a manner similar to that described above with reference to FIG. 3, the resorbable thread (or other resorbable or dissolvable material) is woven (or inserted) into the openings 235 of the bridge elements 234, acting as a temporary spacer to hold the bridge 202 in its elongated state. Resorbable materials are those that, when implanted into a human body, are resorbed by the body by means of enzymatic degradation and also by active absorption by blood cells and tissue cells of the human body. Examples of such resorbable materials are PDS (Polydioxanon), Pronova (Poly-hexafluoropropylen-VDF), Maxon (Polyglyconat), Dexon (polyglycolic acid) and Vicryl (Polyglactin). As explained herein, a resorbable material may be used in combination with a shape memory material, such as Nitinol, Elgiloy or spring steel to allow the superelastic material to return to a predetermined shape over a period of time. When the resorbable thread is dissolved over time by means of resorption, the bridge 202 assumes its shortened state.

With continued reference to FIG. 5, it can be seen that the distal end portion 206 of the bridge 202 extends past the proximal end of the distal anchor 212. The distal end portion 206 is joined to the distal anchor 212 by a pair of links 228. As can be seen in FIGS. 4 and 5, by “embedding” the bridge within the proximal and distal anchors, more expandable elements 234 are provided along the length of the bridge. In one preferred configuration, about 8 to 10 mm of the bridge 202 is embedded within the distal anchor 212. As described above, “overlapping” or “embedding” the bridge into the anchors allows the bridge to contract a greater distance as the resorbable material dissolves. In an advantageous feature of the illustrated embodiment, it will be understood that the improved mitral valve repair implant provides greater effectiveness for treating a defective mitral valve without adding overall length to the implant and without sacrificing the integrity of the anchoring portions.

The connecting links 228 are preferably provided along a longitudinal reinforcement member 230 which extends along the length of the distal anchor 212. The reinforcement member 230 provides a backbone to which the connecting links 230 and expandable cells 240, 242, 244 are connected. The connecting links 230 may be of varying lengths and are preferably configured to allow limited vertical and lateral displacement of the bridge 202 relative to the anchor. This structure provides a “suspension” element allowing the bridge to angulate and align within the coronary sinus semi-independently from the anchor 212. Due to this configuration, the implant is better capable of withstanding the mechanical stresses and strains which occur during placement of the implant within the tortuous anatomy of the coronary sinus.

With continued reference to FIGS. 4 and 5, the proximal and distal anchors 210, 212 are preferably cylindrical in construction and are made from a shape memory material, such as, for example, Nitinol. However, in alternative constructions, the anchors 210, 212 may also be made from any other suitable material, such as stainless steel. The anchors 210, 212 preferably have a bellowed configuration which allows the anchors to transform from a compressed state to an expanded state and from an expanded state to a compressed state. In the illustrated embodiment, the distal anchor 212 comprises a number of adjacent cells 240, 242, 244, each cell comprising a series of zig-zag members. The zig-zag members provide the anchor with the ability to expand and contract. The zig-zag members also provide the anchor with excellent flexibility to conform to the coronary sinus.

As noted above, the proximal and distal anchors 210, 212 each have a compressed state and an expanded state. In the compressed state, the anchors have a diameter that is less than the diameter of the coronary sinus. In this state, the anchors have a substantially uniform diameter of between about 1.5 mm to 4 mm. In the expanded state, the anchors have a diameter that is about equal to or greater than a diameter of the section of a non-expanded coronary sinus to which each anchor will be aligned. Since the coronary sinus has a greater diameter at its proximal end than at its distal end, in the expanded state the diameter of the first anchor 12 is between about 10 mm and 15 mm and the diameter of the second anchor 14 is between about 3 mm and 6 mm.

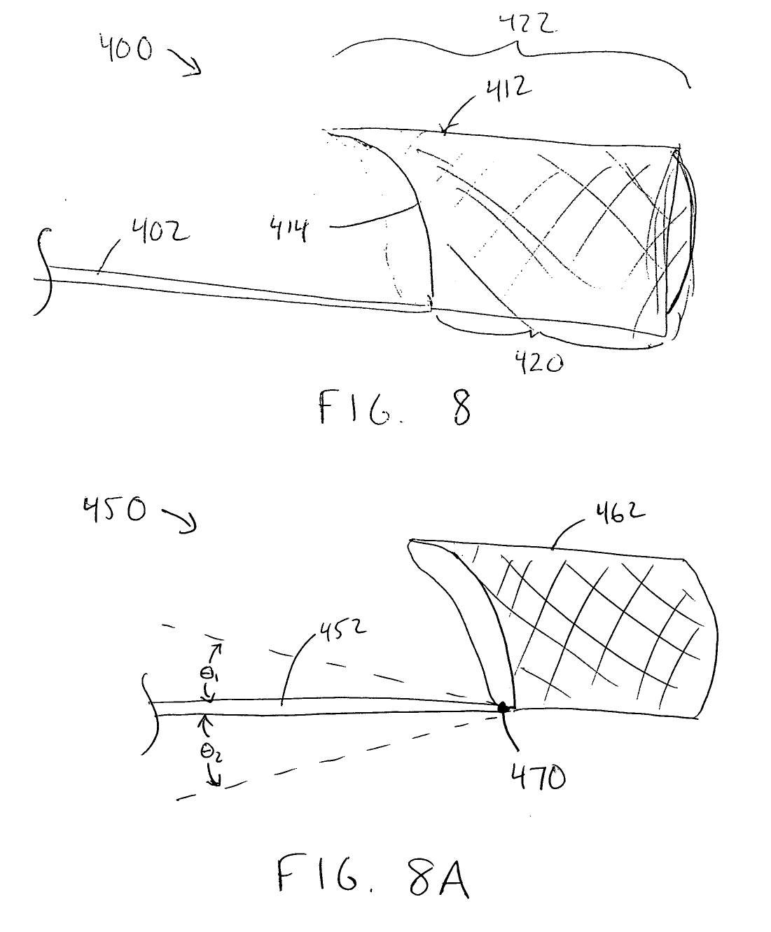

A preferred delivery method will now be described wherein the mitral valve repair implant is delivered to the coronary sinus using a percutaneous approach. Although the delivery method is described using a catheter-based percutaneous approach, it should be appreciated that embodiments of the implant described herein may also be implanted via a surgical procedure. With reference now to FIGS. 4 through 6, the mitral valve repair implant 200 is preferably loaded onto a delivery device 250 before use. The cylindrical anchors 210, 212 are collapsed (e.g., crimped while in a cooled condition) into their compressed state onto an inner tubing 252 such that the inner diameters of the anchors are approximately equal to the outer diameter of the inner tubing. Once the implant 200 has been placed on the inner tubing 252 at a desired location, an outer sheath 254 is advanced over the implant. The orientation of the bridge 202 with respect to the delivery device 250 may be noted using fluoroscopy such that an operator of the delivery device can place the bridge in the desired location within the coronary sinus.

After a patient is prepared, an introducer sheath is preferably inserted into a left or right internal jugular vein or the femoral vein which provides access to the coronary sinus as is generally known in the art. In an alternative delivery method, access to the coronary sinus may be achieved through a subclavian vein. In any case, once the introducer sheath is secured, a guidewire is inserted through the introducer sheath and into the coronary sinus. A guide catheter combined with a dilator is inserted along the guidewire under fluoroscopy until a distal end of the guide catheter is positioned at a desired location in the coronary sinus. The dilator is then withdrawn proximally from the guide catheter. Once the guide catheter has been secured within the coronary sinus, a delivery catheter with the implant 200 mounted thereon is inserted onto the guide catheter and advanced until the implant is in a desired location. With the implant 200 in its desired location, the guide catheter is retracted to expose the section of the delivery system on which the elongate body is mounted. Ensuring that the section containing the implant 200 extends beyond a distal tip of the guide catheter prevents the elongate body from being deployed inside the guide catheter rather than inside the coronary sinus.

Using a sliding button 266 on a handle portion 262 of the delivery device 250, the outer sheath 254 is retracted until the distal anchor 212 is deployed. The relative position and/or displacement of the outer sheath 254 with respect to the inner tubing 252 may be determined by viewing marker bands 264 on the outer sheath and inner tubing under fluoroscopy. Once the distal anchor 212 is deployed, the handle portion 262 and the delivery catheter 260 are pulled proximally to position the bridge 202 of the implant 200 along the anterior wall of the coronary sinus and to eliminate as much slack as possible from the delivery catheter. After the implant 200 has been positioned as desired location, the sliding button 266 is further retracted proximally to expose the bridge 202 and the proximal anchor 210 to the wall of the coronary sinus.

After the delivery catheter 260 has been removed from the patient, a venogram (i.e. an X-ray of a contrast medium filled vein) may be performed in the coronary sinus to ensure the patency of the implant 200. The guide catheter, guidewire, and the introducer sheath may then be removed, leaving the implant in the patient. Over time, the implant reshapes the mitral valve annulus as described above such that the posterior leaflet is pushed toward the anterior leaflet, thereby reducing the gap in the mitral valve.

If desired, an alternative method of operation may also be used with the improved mitral valve repair implant. With reference again to FIGS. 4 and 5, the implant 200 may further comprise proximal and distal eyelets 220, 222. The eyelets are configured such that a mandrel (not shown) may be used for acutely treating the mitral valve. A distal end of the mandrel may be adapted to be releaseably attached to the distal eyelet 222 on the implant. In one exemplary embodiment, the distal end of the mandrel may have a threaded configuration. A proximal end of the mandrel may be adapted to be releasable attached to the proximal eyelet 220 of the implant.

The operation of the implant and the mandrel is as follows. The implant 200 is first stretched to about 150% of its length and the mandrel is inserted into the eyelets 220, 222 to maintain the elongate body in the stretched elongated state. The combination of the mandrel and the implant 200 may then be inserted into the coronary sinus as described above. After the proximal and distal anchors (e.g., stents) are fixed within the coronary sinus, the mandrel may then be manipulated to release the proximal and distal anchors. As a result, the implant 200 contracts, thereby producing a desired shape for reshaping the mitral valve annulus. The shortening effect of the elongate body may be monitored by using fluoroscopy and when the desired effect is reached, the mandrel may be removed from the implant.

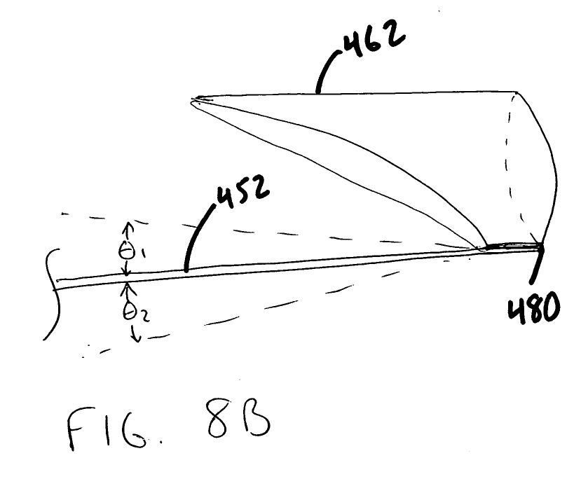

With reference now to FIG. 7, the distal end portion of an alternative mitral valve repair implant 300 is shown. For ease of illustration, only a distal anchor 312 is illustrated. However, the features and aspects described herein are also preferably applied to the proximal anchor. Similar to the embodiment described above with respect to FIGS. 4 and 5, this embodiment includes a bridge 302 which is embedded within the proximal anchor and distal anchor 312. However, in this configuration, the shape-changing structure of the bridge 302 extends further into the anchors. For example, as can be seen in FIG. 7, the bridge 302 extends to the distal end of the distal anchor 312. Accordingly, the bridge is provided with a larger number of expandable elements as compared with the previously described configuration, thereby providing even greater contraction as the resorbable material is resorbed within the body. Accordingly, it will be recognized that the bridge extends along substantially the entire length of the mitral valve repair implant, thereby maximizing the effectiveness of the implant in relation to its overall length. In this embodiment, it can be seen that the bridge is preferably attached to the distal anchor by a plurality of links 328, 330, 332. Because the bridge contracts (i.e., foreshortens) over time, the connecting links 328, 330, 332 are configured with sufficient flexibility to accommodate the axial movement of the bridge relative to the substantially fixed length structure of the anchor 312.

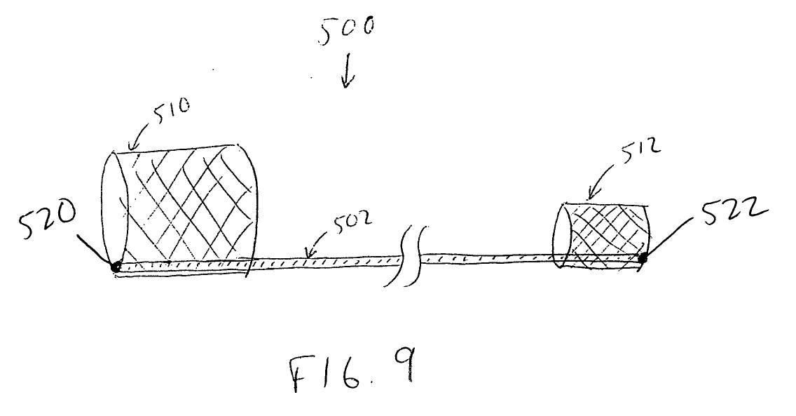

With reference to FIG. 8, the distal end portion of an alternative mitral valve repair implant 400 is illustrated wherein a proximal end of the distal anchor 412 is formed with a curved or angular surface 414 such that the bridge 402 may be attached at a location deeper into the anchor. The structure of the anchor 412 is substantially similar to the anchors described above, and the anchor may be transferred between a compressed state and an expanded state. However, in this variation, a portion of the anchor has been “cut away” such that one end of the anchor has an angled configuration. More specifically, the edge 420 to which the bridge is attached has a shorter length 420 and the opposite edge has a longer length 422. This anchor configuration allows for the anchor to have a length sufficient to provide a secure attachment to the coronary sinus, yet also allows for a longer bridge. As discussed above, the longer bridge translates into a greater degree of contraction, thereby allowing for a more effective treatment of mitral valve insufficiency. The length of the shorter side of the anchor 412 to the length of the longer side may be between about 33% to about 75%. Although not shown, a similar construction is also preferably applied to the attachment of the bridge to the proximal anchor.

With reference to FIG. 8A, a variation of FIG. 8 provides an implant 450 comprising a bridge 452 and a distal anchor 462, wherein the shape-changing member and anchors are capable of rotating relative to each other along at least one axis. For example, as shown in FIG. 8A, the bridge is capable of rotating upward by a first angle θ₁ and downward by a second angle θ₂ relative to the anchor. It has been found that the human anatomy of the coronary sinus has curvatures that would benefit from a more flexible and adaptive connection between the anchor and the bridge. Specifically, a bridge that is capable of pivoting or rotating relative to the anchors provides the bridge with the freedom to follow a more aggressive curvature angle relative to the anchors. By embedding the connection point deeper (e.g. approximately 2 cm) into the anchors, the bridge is provided with even greater flexibility and is less constrained by the lengths of the anchors. It has also been found that pivoting, rotation or articulation of the bridge relative to the proximal and distal anchor advantageously reduces the stress concentrations at the attachment points. It will be appreciated that these features are particularly advantageous when the proximal or distal anchor is deployed in a tight curve within a tortuous coronary sinus. In other words, the take-off angle θ of the bridge relative to the anchors may self-adjust to conform to the particular patient anatomy. FIG. 8B illustrates yet another variation wherein the bridge 452 is fixed to the distal end of the distal anchor 462 at attachment point 480. In this embodiment, the anchors preferably take the form of a cylindrical stent with a portion cut-away. Although the bridge is not rotatably coupled to the anchor, the cut-away shape of the anchor provides the bridge with more bending flexibility and also allows the bridge to move without interference from the anchor.

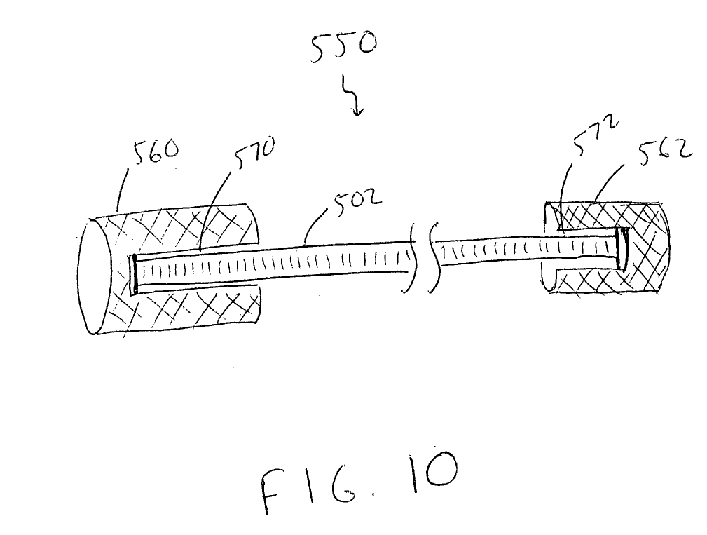

With reference now to FIG. 9, yet another alternative embodiment of an improved mitral valve repair implant 500 comprises an elongate bridge 502, a proximal anchor 510 and a distal anchor 512. The proximal and distal anchors are preferably substantially cylindrically shaped stents. For ease of description, the anchors 510, 512 are shown in the expanded condition. In this embodiment, the bridge 502 extends through the central openings in the proximal and distal anchors 510, 512. The bridge has a proximal end 520 which is fixed to a proximal end of the proximal anchor 510. Similarly, the bridge has a distal end 522 which is fixed to a distal end of the distal anchor 512. In one method of construction, the bridge and the anchors may be integrally formed from a single piece of material. In alternative constructions, the bridge may be attached to the anchors using mechanical connections, welding, adhesives, suturing or any other suitable means of attachment. In any case, the bridge is preferably constructed to contract after deployment and the bridge preferably extends along the entire length of the mitral valve repair implant, thereby maximizing the degree of contraction.

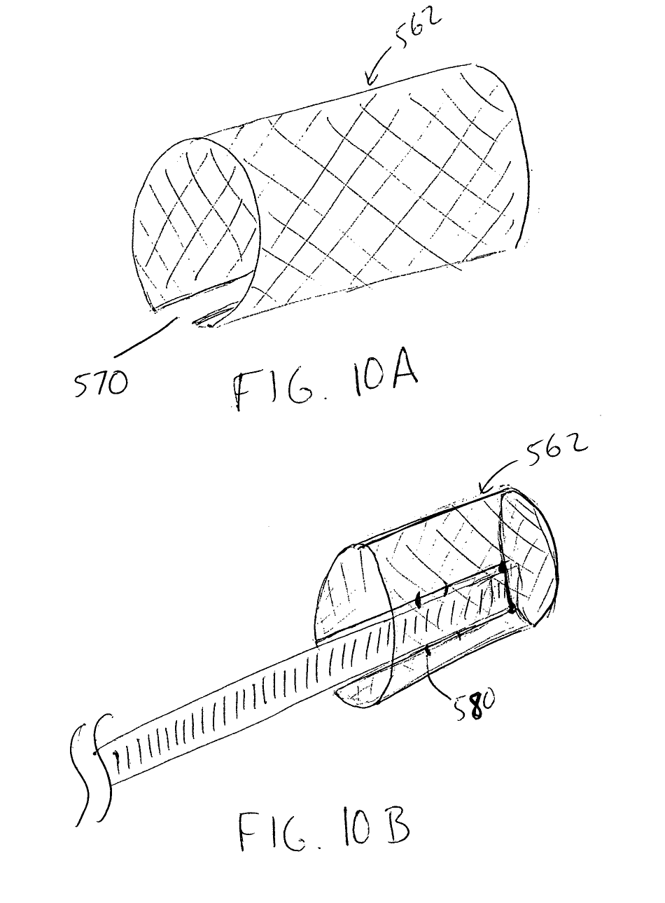

With reference now to FIGS. 10 through 10B, another variation of a mitral valve repair implant 550 comprises a bridge 502, a proximal anchor 560 and a distal anchor 562. In this variation, a slot 570 is provided in the wall of the proximal anchor 560 for receiving a proximal end portion of the bridge 502. Similarly, a slot 572 is provided in the wall of the distal anchor 562 for receiving a distal end portion of the bridge 502. With this construction, it can be seen that the implant 550 is configured such that the entire length of the bridge 502 is situated to extend along the vessel wall, thereby allowing for better tissue in-growth into the bridge over time. In one preferred embodiment, the proximal and distal ends of the bridge 502 are tied to the anchors with suture. Holes may be provided on the bridge for receiving the suture and facilitating the connection to the anchors. In other alternative embodiments, the bridge may be connected to one or both anchors via a non-rigid connection such as, for example a hinge or via connecting loop members. In preferred embodiments, the coupling mechanism would provide unrestrained relative movement between the anchor and bridge along at least one axis. This type of coupling mechanism may be used with any of the embodiments described herein for eliminating stress concentrations at the connection point between the bridge and anchor. With reference to FIG. 10A, the distal anchor in isolation. FIG. 10B illustrates the connection between the bridge 502 and the distal anchor 512. To better ensure a desired alignment between the bridge and the anchors, a portion of the bridge may be slideably attached to the anchors. For example, with reference to FIG. 10B, protrusions 580 may be provided for slidably engaging the bridge to the edges of the slot 570 in the anchor 562.

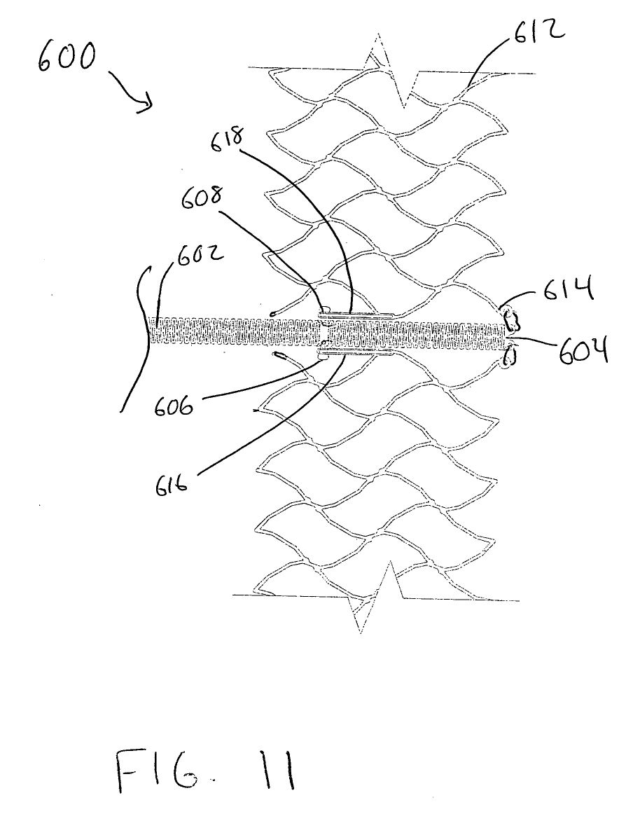

With reference to FIG. 11, yet another configuration of an anchoring mechanism is provided. FIG. 11 illustrates a bridge 602 coupled to a distal anchor 612. In this coupling configuration, the distal end 604 of the bridge is tied to the distal end 614 of the distal anchor via one or more sutures. To facilitate the attachment, holes may be provided along the distal end of the bridge and along the distal end of the anchor for receiving the suture. Proximal to the distal end 604 of the bridge 602, first and second lateral protrusions 606, 608 are provided along the sides of the bridge. The first and second protrusions are sized to be received within first and second slots 616, 618 formed in the anchor. The slidable relationship between the protrusions and slots allows the bridge to move axially relative to the anchor as it contracts.

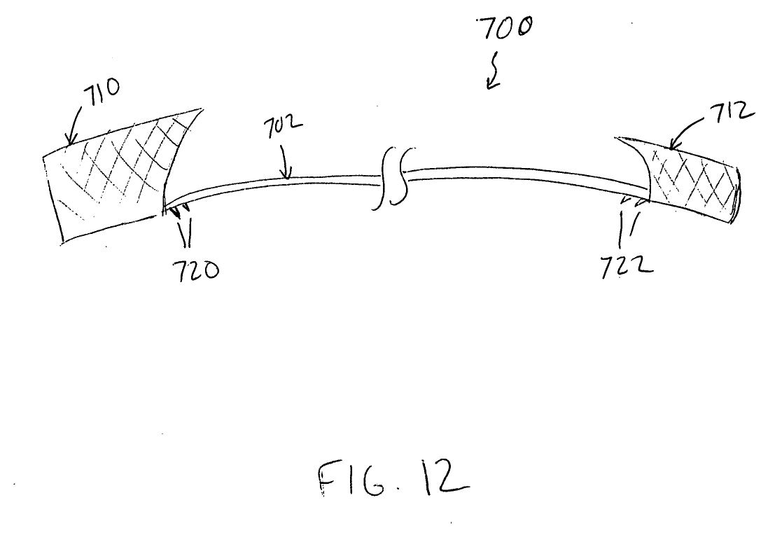

With reference now to FIG. 12, another embodiment of a mitral valve repair implant 700 comprises a bridge 702 and proximal and distal anchors 710, 712 which are similar in many respect to the anchors described above with respect to FIG. 8. However, in the embodiment, tabs or barbs 720, 722 are disposed along the bridge. The barbs are preferably located along the ends of the bridge to further resist relative movement between the ends of the bridge and the wall of the coronary sinus. In one preferred embodiment, two tabs are provided along each end of the bridge for penetrating the tissue of a wall of the coronary sinus. The tabs protrude from the bridge and may be of any shape sufficient to enhance the anchoring and cinching capability of the bridge. For example, the tabs may be triangularly shaped.

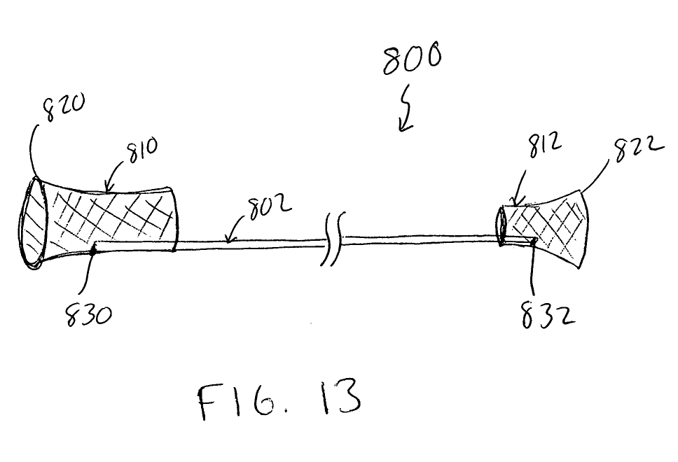

With reference now to FIG. 13, another embodiment of a mitral valve repair implant 800 comprises a bridge 802 and proximal and distal anchors 810, 812. In this embodiment, a proximal region 820 of the proximal anchor 810 has an outwardly tapered configuration such that it flares away from a central axis of the anchor. Similarly, a distal region 822 of the distal anchor 812 has an outwardly tapered configuration such that it flares away from a central axis of the anchor. The flared regions serve to provide additional anchoring capability to the anchors. In one exemplary embodiment, the proximal region of the proximal anchor generally conforms to the shape of the ostium of the coronary sinus. When deployed, the proximal and distal anchors are constrained by the diameter of the coronary sinus. Accordingly, the end regions 820, 822 may not fully expand to the flared (i.e., fully expanded) shape shown in FIG. 13 during actual use.

While the foregoing described the preferred embodiments of the invention, it will be obvious to one skilled in the art that various alternatives, modifications and equivalents may be practiced within the scope of the appended claims.

What is claimed is:

1. An implant for treating mitral valve insufficiency, comprising:
a shape-changing member having a proximal end portion and a distal end portion, the shape-changing member having a first shape and a second shape;
a displaceable material disposed along the shape-changing member for maintaining the shape-changing member in the first shape, the displaceable material being configured to be displaced for allowing the shape-changing member to transition from the first shape to the second shape;
a proximal anchor coupled to the proximal end portion of the shape-changing member; and
a distal anchor coupled to the distal end portion of the shape-changing member;
wherein the proximal end portion of the shape-changing member overlaps with at least a portion of the proximal anchor and the distal end portion of the shape-changing member overlaps with at least a portion of the distal anchor.

2. The implant of claim 1, wherein the shape-changing member is coupled to the proximal and distal anchors by an attachment mechanism selected from a group consisting of suture, wire, and polymer.

3. The implant of claim 1, wherein the shape-changing member is flexibly coupled to the proximal and distal anchors.

4. The implant of claim 1, wherein the shape-changing member and the proximal and distal anchors are integrally formed from a single piece of material.

5. The implant of claim 1, wherein the shape-changing member, the proximal anchor and the distal anchor are constructed separately and joined together to form the implant.

6. The implant of claim 1, wherein the proximal and distal anchors are configured to self-expand.

7. The implant of claim 1, wherein the proximal and distal anchors comprise proximal and distal stents.

8. The implant of claim 7, wherein one end of each stent has a curvilinear shape such that a first wall of the stent has a first longitudinal length and a second wall of the stent has a second longitudinal length which is longer than the first length and wherein the shape-changing member is attached to the first wall.

9. The implant of claim 7, wherein the proximal end portion of the shape-changing member extends through an interior region of the proximal stent and the distal end portion of the shape-changing member extends through an interior region of the distal stent.

10. The implant of claim 9, wherein the shape-changing member is fixedly attached to a proximal end of the proximal stent and to a distal end of the distal stent.

11. The implant of claim 7, wherein longitudinal slots are provided in the proximal and distal stents, the longitudinal slots being configured for receiving the proximal and distal end portions of the shape-changing member.

12. The implant of claim 1, further comprising barbs disposed along the proximal and distal end portions of the shape-changing member, the barbs being configured for engaging tissue after implantation.

13. The implant of claim 7, wherein at least one of the proximal and distal stents has a flared end region.

14. The implant of claim 1, wherein at least a portion of the shape-changing member is slidably coupled to at least one of the proximal and distal anchors.

15. The implant of claim 1, wherein the shape-changing member is rotatably coupled to at least one of the proximal and distal anchors.

16. A medical implant, comprising:
a proximal anchor configured for engagement with an inner wall of a coronary sinus when in a deployed position;
a distal anchor configured for engagement with an inner wall of a coronary sinus when in a deployed position; and
an elongate bridge extending between the proximal and distal anchors, the elongate bridge configured for applying a reshaping force along an annulus of a mitral valve;
wherein the proximal and distal anchors are capable of pivoting relative to the elongate bridge along at least one axis.

17. The medical implant of claim 16, wherein the elongate bridge is formed of a shape-memory material, the bridge being maintained in an elongated state by a resorbable material, the bridge being biased to transition to a contracted state as the resorbable material is resorbed.

18. A medical implant for treating a mitral valve, comprising:
a proximal stent configured for engagement to an ostium of a coronary sinus when in an expanded condition;
a distal stent configured for engagement with an inner wall of a coronary sinus when in an expanded condition; and
an elongate bridge coupled to the proximal and distal stents, the elongate bridge formed of a shape-memory material and having a proximal end portion which overlaps with the proximal stent and a distal end portion which overlaps with the distal stent;
wherein the bridge is configured to contract after the proximal and distal stents are anchored within the coronary sinus such that tension in the bridge provides a reshaping force along an annulus of the mitral valve.

19. The medical implant of claim 18, wherein the bridge has a length which is greater than 90% of a total length of the implant.

20. The medical implant of claim 18, wherein a length of the bridge is substantially equal to a total length of the implant.
